(19) United States
(12) Patent Application Publication
(10) Pub. No.: US 2008/0241256 A1
(43) Pub. Date: Oct. 2, 2008

(54) TARGETED ACTIVE AGENT DELIVERY SYSTEM BASED ON CALCIUM PHOSPHATE NANOPARTICLES

(76) Inventor: Liisa Kuhn, West Hartford, CT (US)

Correspondence Address: CANTOR COLBURN, LLP, 20 Church Street, 22nd Floor, Hartford, CT, 06103, US

(21) Appl. No.: 12/059,398

(22) Filed: Mar. 31, 2008

Related U.S. Application Data
(60) Provisional application No. 60/920,924, filed on Mar. 30, 2007.

(51) Int. Cl. A61K 9/16; A61K 33/24; A61P 35/04

(52) U.S. Cl. 424/489; 424/649; 977/773

(57) ABSTRACT

Calcium phosphate nanoparticle active agent conjugates are described. Specifically, anticancer agent conjugates are prepared which are suitable for targeted active agent delivery to tumor cells and lymphatics for the treatment of cancer and the treatment or prevention of cancer metastasis.

### CROSS-REFERENCE TO RELATED APPLICATIONS

This application claims priority to Provisional Application No. 60/920,924 filed Mar. 30, 2007, the entire contents of which are hereby incorporated by reference.

### TECHNICAL FIELD

This invention relates to targeted drug delivery systems based on calcium phosphate nanoparticles comprising an adsorbed active agent.

### BACKGROUND

Controlled delivery of active agents is particularly desired to provide any number of benefits including targeted delivery of an active agent, reduced number of doses, or reduced severity of side effects. Various technologies have been explored to provide controlled delivery injectable formulations such as liposomes and polymer microspheres. However, there are drawbacks for several current delivery systems. Liposomes are still being modified to meet the requirements of being long-circulating in blood, while at the same time efficiently accumulate and transfer drug in a sustained manner to targeted sites. Biodegradable polymer-based drug delivery systems can often form polymer acidic byproducts or degrading polymer fragments which may adversely affect the active agent they are delivering or the tissues they interact with.

Bioceramics, such as calcium phosphates, including hydroxyapatite, have been examined for use as a carrier for non-viral gene delivery, antigens, enzymes, and proteins. Particularly, calcium phosphate disks and pellets have been examined as potential active agent delivery systems. Due to the low solubility of the hydroxyapatite type of calcium phosphate in physiological conditions, hydroxyapatite remains for long periods after in vivo subcutaneous placement. Thus, large sintered disks and large particle of hydroxyapatite utilized in the previously researched formulations would remain in vivo long after drug release.

Chemotherapy treatments, although beneficial for extending the life span of a cancer patient, are fraught with side effects that limit the dose that can be administered and adversely affects patient quality of life. Current treatment for cancer patients includes systemic chemotherapy and radiation to treat residual cancer remaining after primary tumor removal. Targeted drug delivery systems could provide effective, localized drug delivery (e.g., intratumoral delivery of anticancer agent), thereby minimizing systemic toxicity while allowing for an increase in drug administration.

There remains a continuing need in the art for improved controlled release or targeted release formulations. There also remains a need for improved drug delivery systems to provide localized delivery of active agents, especially chemotherapeutic agents, to reduce the potential for side effects while at the same time providing the therapeutic benefit of the chemotherapeutic. Furthermore, since most common solid tumor cancers metastasize via the lymphatic route there remains a continuing need for lymphatic delivery of anticancer agents.

### BRIEF SUMMARY OF THE INVENTION

In one embodiment, an active agent delivery system comprises a calcium phosphate nanoparticle active agent conjugate comprising an active agent adsorbed onto calcium phosphate nanoparticles, wherein the calcium phosphate nanoparticles are prepared with a dispersing agent; and wherein a calcium phosphate nanoparticle active agent conjugate further comprises a targeting ligand.

In another embodiment, a method to treat or prevent a disease condition in a patient comprises administering to a patient in need thereof an active agent delivery system comprising a calcium phosphate nanoparticle active agent conjugate comprising an active agent adsorbed onto calcium phosphate nanoparticles, wherein the calcium phosphate nanoparticles are prepared with a dispersing agent; and wherein a calcium phosphate nanoparticle active agent conjugate further comprises a targeting ligand.

In yet another embodiment, a method to treat cancer comprises administering to a patient in need thereof an active agent delivery system comprising a calcium phosphate nanoparticle active agent conjugate comprising an active agent adsorbed onto calcium phosphate nanoparticles, wherein the calcium phosphate nanoparticles are prepared with a dispersing agent; and wherein a calcium phosphate nanoparticle active agent conjugate further comprises a targeting ligand.

In another embodiment, a method to treat or prevent cancer metastasis comprises administering to a patient in need thereof an active agent delivery system comprising a calcium phosphate nanoparticle active agent conjugate comprising an active agent adsorbed onto calcium phosphate nanoparticles, wherein the calcium phosphate nanoparticles are prepared with a dispersing agent; and wherein a calcium phosphate nanoparticle active agent conjugate further comprises a targeting ligand.

In another embodiment, a method to treat or prevent cancer metastasis comprises administering to a patient in need thereof an active agent delivery system comprising a calcium phosphate nanoparticle active agent conjugate comprising an active agent adsorbed onto calcium phosphate nanoparticles, wherein the calcium phosphate nanoparticles are prepared with a dispersing agent; and wherein a calcium phosphate nanoparticle active agent conjugate further comprises a targeting ligand; and wherein the calcium phosphate nanoparticle active agent conjugate accumulates in the lymph nodes of the patient.

### BRIEF DESCRIPTION OF DRAWINGS

FIG. 1. illustrates ME-180 tumor growth curves in female BALBc mice illustrating the intratumoral administration of the calcium phosphate/cisplatin conjugate is statistically more effective than the same dose of cisplatin given systemically (Example 1).

FIG. 2. illustrates a graph of percent weight change of mice plotted as a function of time for different treatments: intratumoral calcium phosphate/cisplatin conjugate+radiation and systemic intraperitoneal cisplatin+radiation (Example 1).

FIG. 3. illustrates a graph of platinum concentration as a function of time in mouse plasma: 7 mg/kg intraperitoneal cisplatin or 10 m/kg intratumoral injections of calcium phosphate/cisplatin conjugate (Example 1).

FIG. 4. is a graphic illustration of cisplatin concentration in the popliteal lymph node after footpad injections of the calcium phosphate/cisplatin conjugate compared to footpad injections of free cisplatin (Example 1).

FIG. 5. illustrates a comparison of IC50 values obtained for the cytotoxicity testing of cisplatin released after incubation of the conjugates in PBS; the results indicate the IC50 values of the conjugate are lower than the free cisplatin indicating particulates of CaP carrying cisplatin can overcome drug resistance (Example 1).

FIG. 6. is a graphical illustration of particle size analysis of calcium phosphate nanoparticle/cisplatin conjugates made in the presence of DARVAN redispersed in H₂O at 1 mg/mL concentration (Example 2).

FIG. 7. illustrates an active agent release profile of calcium phosphate nanoparticle/cisplatin conjugates made in the presence of DARVAN (88 ug/mg loading): (a) amount of cisplatin released over time in phosphate buffered saline (PBS), pH=7.4; and (b) cumulative release over time of cisplatin in PBS, pH=7.4 (Example 2).

FIG. 8. illustrates a comparison of IC50 values obtained for the cytotoxicity testing of cisplatin: (a) Free active agent control, (b) cisplatin released in PBS from the calcium phosphate nanoparticles made in the presence of DARVAN/cisplatin conjugate; (c)-(e) are from the direct addition study: (c) Free active agent control, (d) calcium phosphate nanoparticles made in the presence of DARVAN+Free active agent; (e) calcium phosphate nanoparticles made in the presence of DARVAN/cisplatin conjugates particles directly added to the cells; (*) denotes significant difference (P<0.05, Student's T-test) from free active agent control; five replicates per group (Example 2).

FIG. 9. is a graphical illustration of the demonstration of IC50 value determination of calcium phosphate nanoparticle/cisplatin conjugates made in the presence of DARVAN on A2780C is cancer cell lines (Example 2).

### DETAILED DESCRIPTION OF THE INVENTION

Disclosed herein are active agent delivery systems comprising a calcium phosphate nanoparticle active agent conjugate. The active agent is adsorbed to the surface of the calcium phosphate nanoparticlates. The high surface area of the nanoparticles allow for the adsorption of large quantities of an active agent that can then be released in a controlled fashion upon introduction into a patient.

The active agent delivery systems can be used for localized, less toxic active agent therapy delivery, for example the delivery of chemotherapeutic agents. The calcium phosphate nanoparticle active agent conjugates are stable, provide immediate and sustained release, are biocompatible, biodegradable, and have non-toxic and non-acidic degradation products. By targeting the active agent delivery, enhanced active agent efficacy can result from localized active agent application.

As calcium phosphate is biocompatible and will not cause inflammation and soft-tissue calcification, it is suitable for treatment of soft tissue. The high active agent loading capacity of the calcium phosphate nanoparticles means that only milligram quantities or less of the nanoparticles will be needed in therapeutic treatments and would not need to be removed afterwards, even from a soft tissue site.

Also provided herein are methods of treating or preventing cancer metastasis as the calcium phosphate nanoparticles are small enough to travel via the lymphatic system. Furthermore, lymphatic targeted delivery of active agents are provided herein.

The calcium phosphate nanoparticles may substantially comprise hydroxyapatite, a type of calcium phosphate that has a similar chemical structure to bone mineral, and hence has excellent biocompatibility and bioactivity. Although hydroxyapatite has low solubility in physiological conditions, due to the small size of the nanoparticles, they could be resorbed faster, carry more active agent while minimizing the amount of calcium phosphate that is implanted, and allow greater tissue perfusion than traditional calcium phosphate preparations.

The calcium phosphate nanoparticles can easily be formed by wet precipitation methods using inorganic salts such as calcium salts and ammonium salts. For example, solutions of calcium nitrate and sodium bicarbonate/ammonium phosphate can be combined under rapid stirring to provide a calcium phosphate precipitate, which can be isolated and optionally lyophilized. The ratio of Ca to P can be chosen to form hydroxyapatite or amorphous forms.

In one embodiment, the process to prepare the calcium phosphate nanoparticles includes calcinating the calcium phosphate nanoparticles to change the surface chemistry of the nanoparticles and to drive out surface water. It has been determined that surface chemistry can affect active agent loading and in vitro active agent release. The calcinating results in calcium phosphate nanoparticles that adsorb higher concentrations of active agent versus particles that have not been calcinated. The calcinating has also been found to increase the activity of an adsorbed active agent versus those nanoparticles that were not calcinated prior to active agent adsorption.

The calcinating can be performed at a temperature of about 50 to about 350° C., specifically about 100 to about 300° C., more specifically about 150 to about 250° C., and still yet more specifically about 180 to about 220° C. The time of the calcinating step can be about 30 minutes to about 25 hours, specifically about 1 to about 10 hours, and yet more specifically about 4 to about 6 hours.

The calcium phosphate nanoparticles can be prepared to have mean particle size diameters of about 10 to about 20,000 nanometers (nm), specifically about 20 to about 10,000 nm, more specifically about 50 to about 5000 nm, still more specifically about 100 to about 1000 nm, and yet more specifically about 120 to about 500 nm. The size of the calcium phosphate nanoparticles can be determined using known techniques in the art, such as laser light scattering techniques, dynamic light scattering techniques, transmission electron microscopy, atomic force microscopy, scanning electron microscopy, etc.

The calcium phosphate nanoparticles may form micrometer-sized agglomerates. However, as used herein, a system containing agglomerated nanoparticles will still be considered a nanoparticle system if a substantial portion of the particles are free nanoparticles (not agglomerated) and/or the microparticles are agglomerates substantially comprising nanoparticles of calcium phosphate as opposed to uniform microparticles of calcium phosphate. Standard techniques can be used to determine individual crystal size of the particles, including Transmission electron microscopy and X-ray Powder Diffraction.

In one embodiment, the calcium phosphate nanoparticles are not milled to provide the desired particle size.

To prevent particle agglomeration during the synthesis of the nanoparticles, a dispersing agent can be added to the reaction system. In one embodiment, the calcium phosphate nanoparticles are prepared into narrow particle size distributions via a process of adding a dispersing agent at the time of initial crystal formation in the process to prepare the nanoparticles. Such narrow distributions can include a distribution of about 10 to about 50 nm, about 100 to about 1000 nm, and the like.

Suitable dispersing agents for use in preparing the calcium phosphate nanoparticles, for example to stabilize and disperse the nanoparticles in the precipitation solution or to stabilize the nanoparticles once isolated, include polymeric dispersing agents, polyelectrolytes (e.g., poly(allylamine hydrochloride)), surfactants, polysaccharides or carbohydrates (e.g., heparin), amino acids (e.g., L-aspartic acid, lysine, glycine), polyamino acids (e.g., poly-L-lysine), poloxamers (“Pluronic”), gelatin, polyethylene glycols, acrylic-based polymeric salts, or a combination comprising at least one of the foregoing. Exemplary acrylic based polymeric salts include polyacrylic acid salts and polymethacrylic acid salts such as the sodium polyacrylates DARVAN®811, DARVAN®812, and DARVAN® No. 7 commercially available from R.T. Vanderbilt Company, Inc. Norwalk, Conn., USA.

In one embodiment, the dispersing agent for use in preparing the nanoparticles is a sodium polyacrylate having a Mw of about 2000 to about 5000, a sodium polymethacrylate having a Mw of about 2000 to about 5000, or a combination comprising at least one of the foregoing dispersing agents.

The calcium phosphate nanoparticle active agent conjugates (“conjugates”) can be prepared by adsorbing an active agent to the nanoparticle. To form the conjugates, the active agent can be mixed with the calcium phosphate nanoparticles and incubated, optionally in the presence of a pharmaceutically acceptable liquid vehicle.

The calcium phosphate nanoparticle active agent conjugates can be prepared to have mean particle size diameters of about 1 to about 20,000 nm, specifically about 10 to about 10,000 nm, more specifically about 50 to about 5000 nm, and still more specifically about 100 to about 1000 nm.

Size of the nanoparticle has been found to affect active agent loading and in vitro active agent release. In one embodiment, calcium phosphate nanoparticles prepared with a dispersing agent binds more active agent and releases the active agent slower than calcium phosphate nanoparticles prepared in the absence of the dispersing agent.

The present calcium phosphate nanoparticles can be conjugated with a wide variety of active agents or biomolecules due to the versatility of the calcium phosphate nanoparticle structure that is capable of binding both positively and negatively charged molecules through simple adsorption.

Classes of active agents that can be used include, for example, alpha-2 adrenergic agents, analgesics, angiotensin-converting enzyme (ACE) inhibitors, antianxiety agents, antiarrhythmics, antibacterials, antibiotics, anticancer agents, antidepressants, antidiabetics, antiepileptics, antifungal antihelminthics, antihyperlipidemics, antihypertensive agents, antiinfectives, antimalarials, antimicrobials, antimigraine agents, antimuscarinic agents, antineoplastic agents, antiprotozoal agents, antipsychotic agents, antispasmodics, antiviral agents, attention-deficit hyperactivity disorder (ADHD) agents, β-blockers, calcium channel blockers, chemotherapeutic agents, cholinesterase inhibitors, Cox-2 inhibitors, hypnotics, hypotensive agents, immunosuppressants, lipotropics, neuroleptics, opioid analgesics, peripheral vasodilators/vasoconstrictors, sedatives, serotonin receptor agonists, and the like.

Exemplary active agents that can be adsorbed on the calcium phosphate nanoparticles include anticancer agents such as, for example, aminoglutethimide, busulfan, carmustine, chlorambucil, cisplatin, cyclophosphamide, cytarabine, dacarbazine, daunorubicin, diethylstilbestrol, doxorubicin, etoposide, fluorouracil, fluoxymesterone, flutamide, gemcitabine, goseraline acetate, hydroxyprogesterone, hydroxyurea, leuprolide, lomustine, mechlorethamine, medroxyprogesterone acetate, megestrol acetate, melphalan, mercaptopurine, methotrexate, paclitaxel, prednisone, procarbazine, tamoxifan, testosterone propionate, thioguanine, vinblastine, vincristine, vindesine, vinorelbine, a pharmaceutically acceptable salt thereof, and a combination comprising at least one of the foregoing anticancer agents. Combination therapies of two or more anticancer agents are fully contemplated in the present systems.

Besides active agents, various other agents can be adsorbed on the calcium phosphate nanoparticles to improve cellular uptake, to modify active agent release, for combination therapy, and the like. Exemplary additional agents include imaging agents. The calcium phosphate nanoparticles can be loaded with additional factors in a layer-by-layer adsorption technique to form a multi-functional nanoparticle. For example, the calcium phosphate nanoparticles, optionally prepared with a dispersing agent, can further be modified with an additional stabilizing polymer, an imaging agent, a small molecule active agent, an antibody, and/or a targeting ligand, each being adsorbed on the nanoparticles in a separate step or combined and adsorbed in a single step.

Exemplary targeting ligands include folic acid, an antibody, vascular endothelial growth factor (“VEGF”), a vitamin, a protein, an amino acid, polyamino acid, combinations thereof, and the like.

Optionally, the calcium phosphate nanoparticle or calcium phosphate nanoparticle active agent conjugate, with or without an additional agent, can be modified with an additional layer of calcium phosphate by suspending the calcium phosphate nanoparticle or conjugate in a solution of calcium and phosphate. The resulting particulates can further be modified with active agent, additional agent, or additional layer of calcium phosphate and the like.

In an exemplary embodiment, aquated cisplatin is adsorbed to the high surface area of calcium phosphate nanoparticles through electrostatic interactions in chloride free solutions. It is believed that the cisplatin is released from the nanoparticles through an ion exchange mechanism involving chloride ions and the low pH tumor environment.

The calcium phosphate nanoparticle active agent conjugates can be formulated into injectable controlled-release formulations. The conjugates can be administered to a patient as an injectable through a needle and syringe, a cannula, or other suitable means. Various routes of administration include subcutaneous injection, intradermal injection, intratumoral injection, peritumoral injection, intramuscular injection, intravenous injection, and the like.

The injectable controlled-release formulation can further comprise a liquid vehicle and optional additives. The liquid vehicle for use to prepare the injectable controlled-release formulation includes any pharmaceutically acceptable liquid, for example water, saline, aqueous phosphate solutions (e.g., sodium phosphate), isotonic salt buffer solutions (phosphate, acetate, citrate), serum, dimethylsulfoxide, an alkyl alcohol, or a combination comprising at least one of the foregoing liquid vehicles. The conjugates can be dispersed in the liquid vehicle under aseptic conditions. The amount of liquid vehicle used to prepare the injectable formulation can be an amount to result in a suitable viscosity for injection through standard needles and cannulas.

The optional additives may include, for example, a pharmaceutically acceptable stabilizer, pH adjusting agent (e.g., hydroxides, carbonates, mineral acids, organic acid, etc.), viscosity adjusting agents (e.g., water soluble or hydrophilic polymers such as cellulosic polymers, polysaccharides, etc.), or a combination comprising at least one of the foregoing.

The calcium phosphate nanoparticle active agent conjugate does not form a hardenable cement.

Depending upon the active agent present in the conjugate, the calcium phosphate nanoparticle active agent conjugate can be used to treat a wide variety of disease conditions or disorders by the administration of a therapeutically effective amount of the active agent in the form of a calcium phosphate nonoparticle active agent conjugate. An “effective amount” or a “therapeutically effective amount” of an active agent means a sufficient amount of the active agent to provide the desired effect. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

In one embodiment, the calcium phosphate nanoparticle chemotherapeutic or anticancer agent conjugate can be used to treat cancer or to treat or prevent cancer metastasis. Specifically, the conjugate can be used to treat various cancers including, for example, head and neck cancer, breast cancer, melanoma, prostate, or cervical cancer by appropriate selection of the active agent.

Additional exemplary treatments will now be described. In one embodiment, the conjugates can be administered for the treatment of solid tumors by locally (e.g., intratumoral, peritumoral, or in the surgical site after tumor resection, etc.) injecting the conjugates to the patient. The application intratumorally may shrink the primary tumor prior to surgical removal resulting in tissue sparing approaches. The local administration can be used as a local radiopotentiator that may reduce the combined toxicities of chemoradiotherapy. Application locally after surgical resection of the primary tumor may be used to prevent local recurrence. The conjugate can be applied interstitially after or before primary tumor removal to treat possible metastases in draining lymph nodes and associated lymphatics. The system can be applied intraperitoneally with or without adding a targeting ligand for ovarian cancer metastasis. The system can be administered systemically with a targeting ligand such as folic acid or an antibody to allow active agent accumulation in cancer cells through leaky tumor vasculature.

The potential benefits to the cancer patient using the described system includes a reduction of active agent side effects due to localization of the toxic chemotherapy, thereby providing an improved quality of life during and after chemotherapy. The system can provide increased active agent efficacy as the cancer cells are subjected to much higher active agent doses. As the system provides sustained release of the active agent, fewer chemotherapy treatments will be needed. Tissue-sparing approaches can be utilized after neoadjuvant use, smaller margins required due to tumor shrinkage. Administration can result in the avoidance of lymphatic disorders that occur as a result of therapeutic surgery or radiation for cancer in which the lymph nodes are removed or damaged.

In one embodiment, the system can be used for lymphatic targeted active agent delivery. For example, as many cancers disseminate through the lymphatic route, the system described herein can not only treat the primary tumor, but can target the lymphatic tissue to destroy metastatic cells. The nanoparticle conjugates can be injected into the patient (e.g., by subcutaneous injection), and move through the draining lymphatic system, thereby targeting metastasizing cancer cells which also traverse the lymphatics. The size of the nanoparticles can be selected to allow them to travel the lymphatic system and be trapped at draining nodes to allow for local high concentrations of chemotherapy in the closest lymph nodes to the tumor.

Size of the nanoparticle has an influence of lymph node accumulation as smaller particles accumulate more quickly than larger particles. For lymphatic delivery of an active agent, the size of the calcium phosphate nanoparticle conjugates, which may be in the form of agglomerated particles, can be about 10 micrometers or less, specifically about 1 micrometer or less, more specifically about 500 nanometers or less, and yet more specifically about 250 nanometers or less. Exemplary ranges include about 10 nanometers to about 10 micrometers, specifically about 25 nanometers to about 1 micrometer, more specifically about 50 nanometers to about 500 nanometers, and yet more specifically about 100 nanometers to about 250 nanometers.

In one embodiment, after injection into a patient, elevated levels of the anticancer agent is present in the closest draining lymph node to the injection site greater than 24 hours after injection, specifically greater than 48 hours after injection, and more specifically greater than 1 week after injection.

In one embodiment, the calcium phosphate nanoparticle conjugate comprises adsorbed aquated cisplatin (the Cl ions of the cisplatin are replaced with water). Upon injection into the body of the patient, the release of cisplatin occurs when chloride ions are present, releasing cisplatin from the calcium phosphate via an ion exchange mechanism.

In one embodiment, the calcium phosphate nanoparticle active agent conjugate can include a targeting ligand leading to selective cancer cell uptake and active agent release.

Current treatment for cancer patients includes systemic chemotherapy and radiation to treat residual cancer remaining after primary tumor removal. The disclosed nanoparticle calcium phosphate anticancer agent conjugate can be used to replace systemic chemotherapy dose and removal of tumor or lymph nodes or could be used in combination with lower doses of systemic chemotherapy.

In another embodiment, the method of treating cancer or metastasis can occur without substantially affecting the quality of life of the patient, due to decreased side effects of the chemotherapy which may include loss of patient body mass nephrotoxicity (if cisplatin is delivered) or white blood cell decrease, or other side effects associated with the particular chemotherapy agent being delivered on the calcium phosphate nanoparticles.

The nanoparticle calcium phosphate anticancer agent conjugate can be used as a combination therapy with radiotherapy, surgery, systemic chemotherapy, or a combination comprising at least one of the foregoing.

In one embodiment, the calcium phosphate nanoparticle active agent conjugate overcomes drug resistance, as compared to the active agent used alone, via intracellular conjugate uptake. Specifically, the conjugate is prepared with an anticancer agent. It has been observed that chemotherapy agents when enclosed in liposomal delivery systems increases the efficacy of the active agent against drug resistant cancer cells. In vitro studies have shown that cisplatin conjugated to hydroxyapatite nanocrystals is more effective (has increased cytotoxicity) than pure cisplatin against the A2780cis human ovarian cancer cell line which is cisplatin resistant. Mere addition of calcium phosphate particles to a cisplatin solution does not increase the active agent efficacy. Conjugation of the active agent to the surface of the calcium phosphate nanoparticles provides the increased efficacy.

The calcium phosphate nanoparticle active agent conjugates can be formulated to release the active agent in vivo over a period of about 1 day to about 3 months, specifically about 5 days to about 1 month, and more specifically about 7 days to about 14 days.

In one embodiment, an active agent delivery system comprises a calcium phosphate nanoparticle active agent conjugate comprising an active agent adsorbed onto calcium phosphate nanoparticles; and wherein the nanoparticles have been calcinated at about 50 to about 350° C., specifically about 100 to about 300° C., and more specifically about 180 to about 220° C. prior to the adsorption of the active agent. The time for calcinations is about 30 minutes to about 25 hours, specifically about 1 hour to about 10 hours, and more specifically about 4 to about 6 hours. The prepared calcium phosphate nanoparticles are hydroxyapatite prepared by a precipitation process, wherein the calcium phosphate nanoparticles have a mean particle diameter of about 10 to about 100 nanometers, specifically about 100 to about 300 nanometers. Exemplary active agents include an alpha-2 adrenergic agent, an analgesic, an angiotensin-converting enzyme (ACE) inhibitor, an antianxiety agent, an antiarrhythmic, an antibacterial, an antibiotic, an anticancer agent, an antidepressant, an antidiabetic, an antiepileptic, an antifungal antihelminthic, an antihyperlipidemic, an antihypertensive agent, an antiinfective, an antimalarial, an antimicrobial, an antimigraine agent, an antimuscarinic agent, an antineoplastic agent, an antiprotozoal agent, an antipsychotic agent, an antispasmodic, an antiviral agent, an attention-deficit hyperactivity disorder (ADHD) agent, a β-blocker, a calcium channel blocker, a chemotherapeutic agent, a cholinesterase inhibitor, a Cox-2 inhibitor, a hypnotic, a hypotensive agent, an immunosuppressant, a lipotropic, a neuroleptic, an opioid analgesic, a peripheral vasodilator/vasoconstrictor, a sedative, or a serotonin receptor agonist. Specifically the active agent is aminoglutethimide, busulfan, carmustine, chlorambucil, cisplatin, aquated cisplatin, cyclophosphamide, cytarabine, dacarbazine, daunorubicin, diethylstilbestrol, doxorubicin, etoposide, fluorouracil, fluoxymesterone, flutamide, gemcitabine, goseraline acetate, hydroxyprogesterone, hydroxyurea, leuprolide, lomustine, mechlorethamine, medroxyprogesterone acetate, megestrol acetate, melphalan, mercaptopurine, methotrexate, paclitaxel, prednisone, procarbazine, tamoxifan, testosterone propionate, thioguanine, vinblastine, vincristine, vindesine, vinorelbine, a pharmaceutically acceptable salt thereof, or a combination comprising at least one of the foregoing anticancer agents.

The following illustrative examples are provided to further describe how to make and use the nanoparticles and conjugates and are not intended to limit the scope of the claimed invention.

### EXAMPLES

### Example 1

### Preparation of Calcium Phosphate Particles; Calcinated Particles

Calcium phosphate particles were synthesized by the rapid mixture of two solutions: calcium nitrate and sodium bicarbonate/ammonium phosphate. The calcium nitrate solution is prepared by the addition of 500 ml of deionized distilled water to 42.07 g of calcium nitrate tetrahydrate. The sodium bicarbonate/ammonium phosphate solution is prepared by the combination of 80 g of ammonium phosphate dibasic, 40 g of sodium bicarbonate and 1 L of deionized distilled water. The calcium nitrate solution is poured into the sodium bicarbonate/ammonium phosphate solution and let to mature at 25° C. for 7 days. The precipitate formed was then filtered and lyophilized for 3-4 days. Upon completion of lyophilization, the crystals where then calcinated for 5 hours at 200° C. and finally sieved to obtain crystals smaller than 45 μm.

The structure and composition of the calcium phosphate particles were confirmed as pure hydroxyapatite by peak matching x-ray diffraction data and Fourier transform infrared spectras to hydroxyapatite standards. The calcium phosphate particles are sparingly soluble at neutral pH and dissolves at acidic pH. The mean particle size was 7.5 μm by light scattering and the zeta potential was −7.58 mV. A transmission electron micrograph reveals the substantial presence of the nanoparticles less than 100 nm in size.

### Preparation of Calcium Phosphate Particle Active Agent Conjugate; Cisplatin.

Aquated cisplatin was used to fully maximize binding to the calcium phosphate crystals. It was prepared by the addition of 2 moles of silver nitrate (AgNO₃) to 1 mole of cisplatin, which is then mixed for 12-24 hours and protected from light. The silver nitrate was removed thorough a process of filtration and centrifugation, and active agent concentration determined through platinum analysis.

Calcium phosphate particle cisplatin conjugates were prepared by incubating the calcium phosphate particles of Example 1 with aquated cisplatin for 4 hours. Conjugates with active agent loadings ranging up to 80 μg cisplatin/mg calcium phosphate were prepared by varying the concentration of the aquated cisplatin solution and length of time of the binding reaction or the reaction temperature. Conjugated particles were collected by centrifugation and lyophilized. The structure and composition of the calcium phosphate particle conjugate was confirmed as pure hydroxyapatite by peak matching x-ray diffraction data and Fourier transform infrared spectra to hydroxyapatite standards. Active agent loading was determined through platinum analysis by atomic absorption spectroscopy. The conjugate was sterilized by e-beam irradiation, stored at room temperature and protected from light. Conjugates were typically used within one month of manufacture and reconstituted into an injectable paste by the addition of 10 mM potassium phosphate buffer. ¹⁹⁵Pt NMR analysis on extracts of cisplatin released from the conjugate revealed that the released active agent has the same structure as a standard cisplatin solution indicating the active agent has not been altered by the adsorption/release process.

### In Vitro Study; Cisplatin Conjugate

In one set of in vitro cytotoxicity activity studies, the cisplatin-resistant A2780cis human ovarian carcinoma cell line was used. A2780cis human ovarian carcinoma cell line (Sigma, 93112517) was cultured according to supplier's descriptions. IC50 values (50% inhibitory concentration) were determined with the CellTiter96® AQueous One (Promega) colorimetric proliferation assay. The CellTiter96® AQueous One (Promega) colorimetric proliferation assay was used to determine the IC50 value (50% inhibitory concentration) evaluated from 12 two-fold dilutions of: (a) cisplatin (200 μg cisplatin/mL) in 0.9% saline (free active agent), (b) 1.5 mg of conjugates in 1.75 ml PBS, (c) 1.5 mg of hydroxyapatite in 1.75 ml of cisplatin solution. Control wells containing the hydroxyapatite only were used since the hydroxyapatite interferes with the CellTiter96 assay. Absorbance values from the hydroxyapatite only were subtracted from the conjugate data in order to determine the IC50 values. The cytotoxicity assay was conducted as follows: twenty-four hours after seeding 2000 A2780cis cells in 50 ml of media on 96 well plates, 50 ml PBS, or PBS with drug or conjugates was added to the wells.

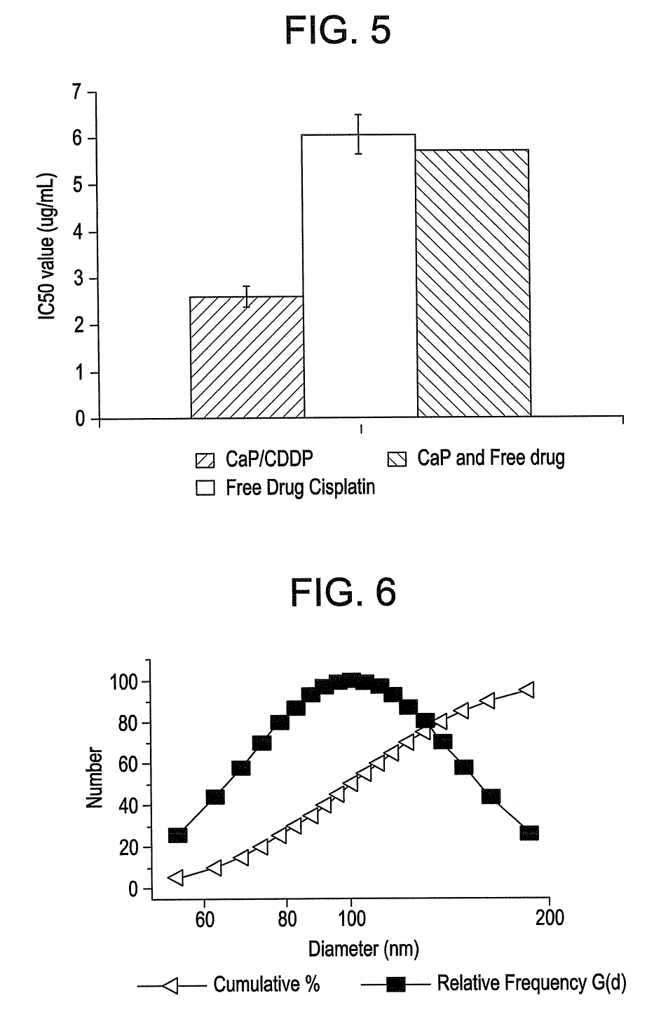

The IC50 values obtained were (in ug/ml): (a) free cisplatin 6.07±0.226, (b) CaP/cisplatin 2.6±0.42, (c) CaP and free cisplatin 5.75 (FIG. 5). The significantly lower IC50 value of the CaP/cisplatin group indicates that the CaP/cisplatin can overcome drug resistance in vitro. In separate experiments it was found that there were no statistically significant differences between the IC50 values of the extracts of cisplatin released from the CaP/cisplatin and the free active agent, indicating no loss of active agent potency due to the adsorption/release process and no toxic compound release from the particle.

### In Vivo Study; Cisplatin Conjugate

Chemoradiotherapy of ME-180 Tumors with intratumoral calcium phosphate particle/cisplatin conjugate was performed. Primary tumors were initiated on the right flank in seventy athymic Ncr-nu/nu mice, 5-7 weeks old, by intradermal injections of 2.25×10⁶ ME-180 cells, a human cervical cancer line. Six treatment groups were established for the study and included an untreated control, 7 mg/kg intraperitoneal (IP) cisplatin, and intratumoral (IT) calcium phosphate particle/cisplatin conjugate at a dose of 10 mg/kg. These three treatment groups were repeated in combination with a single dose of radiation at 8Gy (Varian 2100C), for a total of six treatment groups. Mice were entered into treatment groups once the intradermal ME-180 tumors were 100 mm³±10% in size. A minimum of 5 mice was placed into each group. The conjugate was injected via an 18-gauge needle directly into the intradermal tumor. Tumor length and width were measured daily and the volume determined (Tumor volume=(width)²×length×0.4). Mouse weight was also recorded daily as weight loss is an indicator of cisplatin side effects. Statistical analysis, a one-way ANOVA and Newman-Kuels Comparison Test, was applied to the tumor volumes on day 15 after treatment.

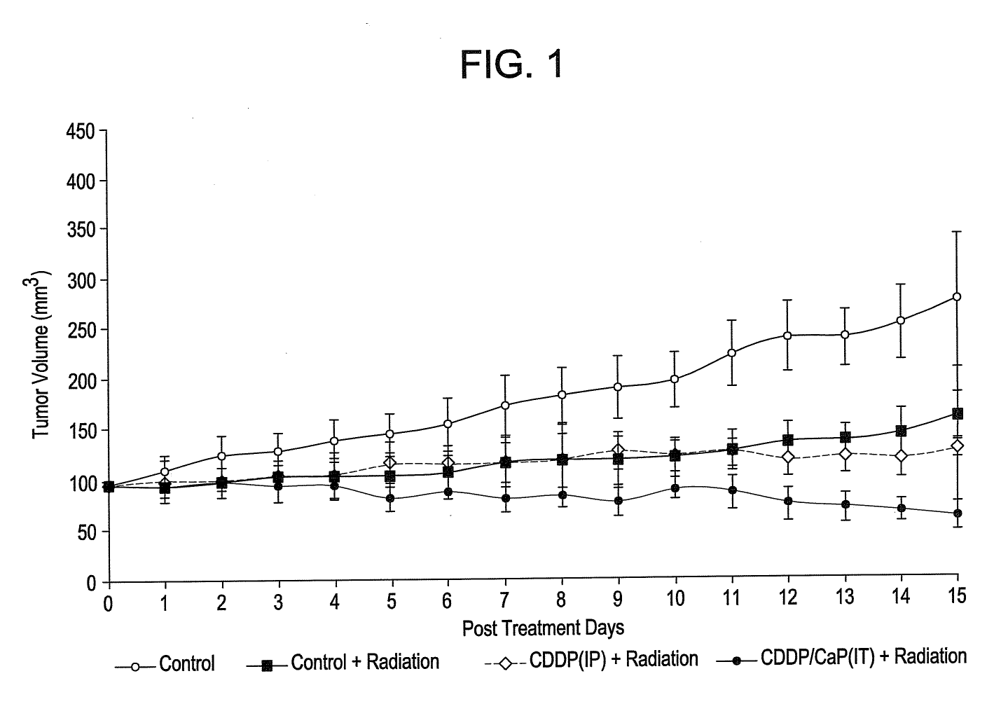

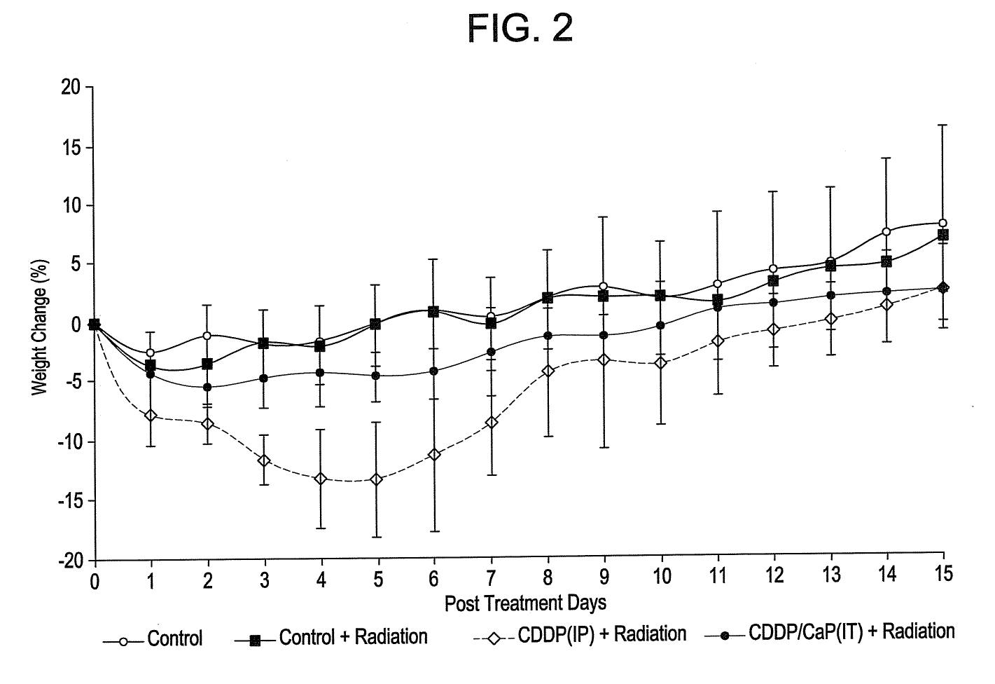

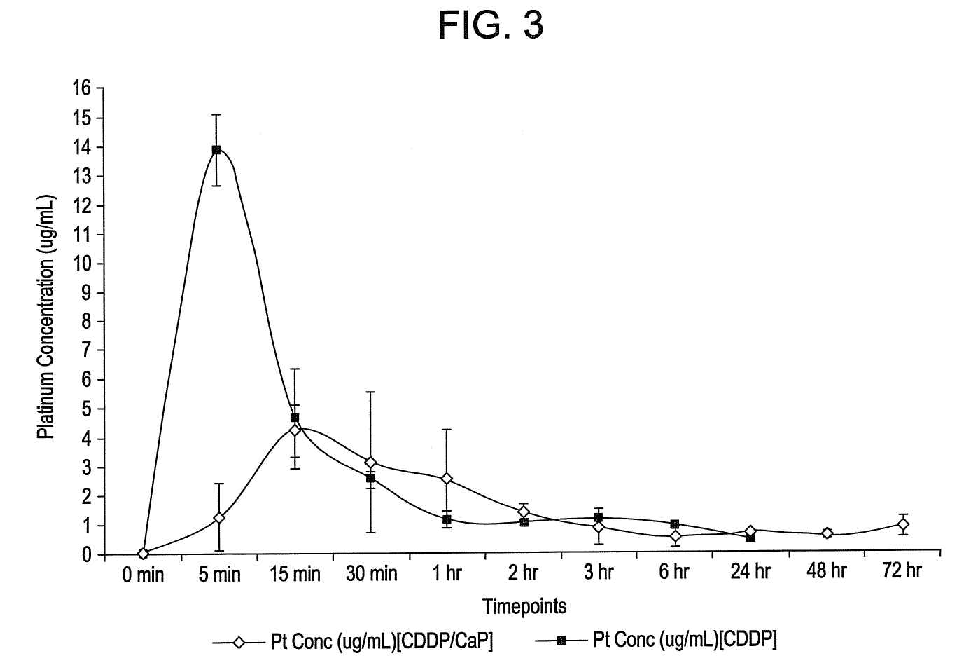

Results indicate that the calcium phosphate particle/cisplatin conjugate (IT)+Radiation treatment was the most effective of all groups and significantly different (p<0.05) than radiation alone (FIG. 1). More of the tumors completely regressed with calcium phosphate particle/cisplatin conjugate (IT)+Radiation (2/5) than cisplatin(IP)+Radiation (1/6). In the treatment groups without radiation, calcium phosphate particle/cisplatin conjugate (IT) was shown to be very significantly different (p<0.01) than cisplatin(IP) indicating intratumoral active agent application is more effective than systemic active agent administration. In fact, the calcium phosphate particle/cisplatin conjugate (IT) treatment alone without radiation was equally effective as the clinical standard of care for cervical cancer tumors: cisplatin(IP)+Radiation. Weight loss data indicates that delivery of cisplatin via the calcium phosphate particle conjugate is a means of reducing the combined toxicities of chemotherapy and radiation (FIG. 2). In previous studies calcium phosphate particle only was included as a treatment group. No tumor inhibition was observed with calcium phosphate particle only. To support the weight loss data, a mini-PK study was conducted that proves that intratumoral injections of calcium phosphate particle/cisplatin conjugate prevent the peak active agent plasma levels observed with intraperitoneal injections of cisplatin (FIG. 3).

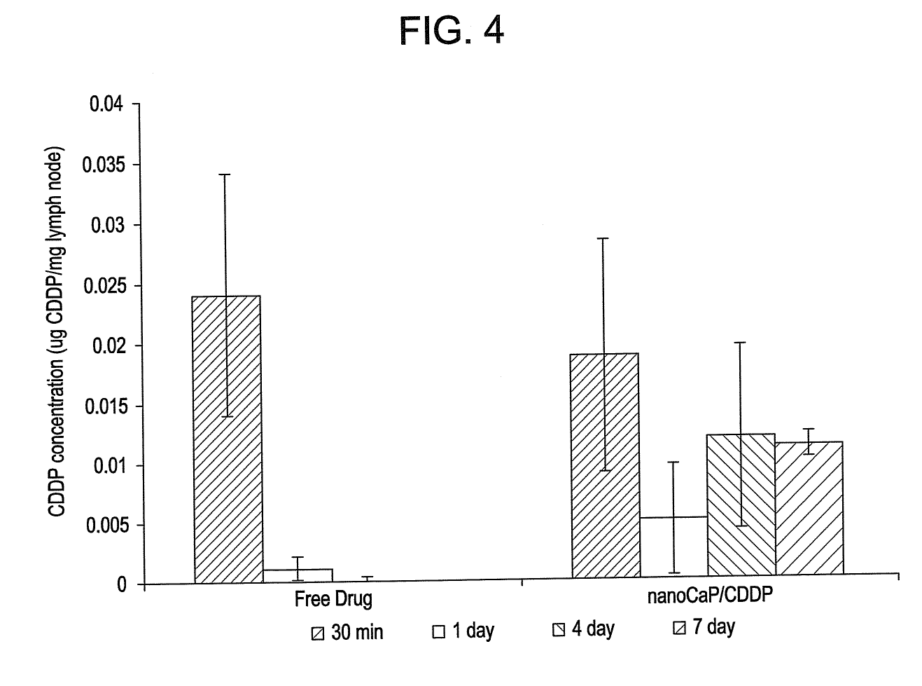

Lymph node accumulation studies were conducted using nine BALB/c mice, 5-7 weeks old injected with 20 μl of calcium phosphate particle/cisplatin conjugate (40 mg/ml) in both rear footpads. Six control mice received 20 μl footpad injections of cisplatin solution only. Draining lymph nodes, as determined by lymphazurin injections (popliteal, inguinal, and lumbar) were collected at 30 min, 1 day, 4 day and 7 days and analyzed for Pt content by graphite furnace atomic absorption spectroscopy. The data for the popliteal nodes, which is the first draining node is shown in FIG. 4. Use of calcium phosphate particle/cisplatin conjugate leads to sustained elevated levels of cisplatin in the draining nodes. For example, calcium phosphate particle/cisplatin conjugate leads to sustained cisplatin levels in the draining popliteal node for at least one week, unlike free cisplatin, which is barely detected after one day. Such results indicate that the conjugate may be more effective than free active agent against lymph node metastases.

### Example 2

### Preparation of Calcium Phosphate Nanoparticles; Particles Prepared in the Presence of a Dispersing Agent

Calcium phosphate nanoparticles were synthesized by precipitation from the addition of equal volumes of a 30 mM Ca(NO₃)₂ solution and a 30 mM K₂HPO₄ solution which are both filtered through 0.1 μm filtration device (Millipore, Boston, USA) separately, followed by immediate addition of 1.67 (v/v) % of 0.2 μm filtered DARVAN®811 (sodium polymethacrylate, M_{W}=3,300, R.T. Vanderbilt Company, Inc. Norwalk, Conn., USA) as a dispersing agent. All reagents are ACS grade and purchased from Sigma Chemical Co., (St. Louis, Mo.), unless noted otherwise. After 1 hr stirring, a pellet of calcium phosphate nanoparticles was collected by centrifugation at 12,000 rpm (20,076 g) for 30 minutes. Before conjugate formation, the calcium phosphate nanoparticle pellet was redispersed in ultrapure H₂O as a wash step, and then collected by centrifugation at 12,000 rpm for 30 minutes. Stably dispersed, nanoparticles of calcium phosphate were obtained by the method of adding DARVAN 811 immediately after precipitation.

### Preparation of Calcium Phosphate Nanoparticle Active Agent Conjugate; Cisplatin

Cisplatin (Sigma Chemical Co., St. Louis, Mo.) was bound to calcium phosphate nanoparticles prepared in Example 2 above by using the aquated form of cisplatin. Aquated cisplatin was prepared by reacting 90 mM AgNO₃ solution with cisplatin solution (about 1000 μg/mL) at a 2:1 molar ratio. The reaction mixture was placed on a thermal rocker (Lab-Line®, model 4637) for 12-24 hrs and kept protected from light. The silver chloride precipitate was removed by several centrifugation steps at 3000 rpm (1000 g) for 20 min. The remaining supernatant was filtered through a 0.2 μm filter. The final concentration of aquated cisplatin was determined by Pt analysis using an Atomic Absorption Spectrophotometer (AAS) (Model 5100, Perkin Elmer, Shelton, Conn., USA).

The conjugate was formed by adding 0.625 mL of 20 mM potassium phosphate buffer (KPB, pH=6) to 31.55 mg of a wet calcium phosphate nanoparticle pellet (which corresponds to 5 mg of dry CaP as determined by oven drying), and sonicating for 10 seconds. Aquated cisplatin (0.625 mL with initial binding cisplatin concentration C₀) was added, and the sample was put in a thermorocker at 37° C., speed 5 (LAB-LINE® thermorocker, Model 4637, Barnstead Thermolyne, IL, USA) for 4 hrs. The conjugates thus formed were centrifuged at 12,000 rpm (20,076 g) for 30 min. The supernatant, which contained unbound cisplatin, was decanted and measured for final binding supernatant cisplatin concentration (C_{f}) by AAS. The pellet was washed with 0.25 mL 10 mM KPB buffer and centrifuged at 12,000 rpm (20,076 g), 30 min. The supernatant from this KPB wash was decanted and measured by AAS to determine KPB wash supernatant cisplatin concentration (C_{KPB}). This pellet was rinsed with 0.21 mL of 0.9% NaCl solution for 30 min. on the thermorocker (37° C., speed 5) after brief sonication. The sample was centrifuged again at 12,000 rpm for 30 min. to collect the calcium phosphate nanoparticle/cisplatin conjugates. The supernatant was decanted and measured for saline wash supernatant cisplatin concentration (C_{w}). The active agent loading was calculated by the following equation:

μg adsorbed cisplatin/mg of CaP=(C₀*V₀−C_{f}*V_{f}−C_{KPB}*V_{KPB}−C_{w}*V_{w})/mg CaP Eq. 1

where V₀, V_{KPB}, and V_{w} are the volume of initial aquated cisplatin, 10 mM KPB buffer, and NaCl used, respectively. Active agent loading efficiency as defined by μg adsorbed cisplatin/μg cisplatin in the starting solution was also calculated. As an alternative means of determining active agent loading, known quantities of the conjugates were fully dissolved in 0.1N HCl and the solution analyzed for Pt content. Aquated cisplatin was simply and efficiently adsorbed to the surface of the nanoparticles through electrostatic interactions.

Three batches of dispersed conjugates were synthesized aseptically for the different studies. Volumes of the precipitation solutions were varied proportionally depending on the yield of conjugates required. The active agent loading of the conjugates was controlled by changing the initial aquated cisplatin concentration (C₀). The active agent loading of conjugates used for in-vitro active agent release study was 88 μg/mg by using 900 μg/mL aquated cisplatin and the active agent loading efficiency was 0.78. The active agent loading of the conjugates used for cytotoxicity test was 35 μg/mg by using 552 μg/mL aquated cisplatin and active agent loading efficiency was 0.5. A lower active agent loading was selected for cytotoxicity testing so that any possible toxic extracts of the calcium phosphate nanoparticle would be present in higher concentrations. Pilot direct addition studies showed that it was not possible to get an IC50 value by direct addition of conjugates unless the active agent loading was much higher. The active agent loading of the conjugates used for the direct addition cytotoxicity study was hence 112 μg/mg obtained by using 1052 μg/mL aquated cisplatin. The active agent loading efficiency was 0.85.

### Physical and Chemical Characterization of Calcium Phosphate Nanoparticle Prepared in the Presence of a Dispersing Agent and Calcium Phosphate Nanoparticle/Cisplatin Conjugates Prepared Therefrom

Samples were prepared for transmission electron microscopy (TEM) by dispersing calcium phosphate nanoparticle/cisplatin conjugates prepared in the presence of a dispersing agent in ultrapure H₂O at about 1 mg/mL concentration with an Ultrasonic 1000 L Cell Disruptor (Ultrasonic Power Corporation, IL, USA) for 1 minute with the sample on ice. One drop of this liquid was immediately transferred by a micropipette to a 3 mm diameter Formvar coated copper TEM grid and slowly evaporated to dryness. The samples on the TEM grid were analyzed using a 100 cx JEOL TEM at 80 kV in brightfield (BF) modes.

TEM images showed that the conjugates prepared in the presence of a dispersing agent are spherical and well dispersed. FIG. 6 illustrates particle size analysis of calcium phosphate nanoparticle/cisplatin conjugates redispersed in H₂O at 1 mg/mL concentration. The mean particle size of the nanoparticles precipitated with DARVAN® 811 before conjugation with cisplatin was 129±133 nm (50% below 125.4 nm, 90% below 181.3 nm), and the zeta-potential=−45.59 mV. The size and zeta-potential slightly decreased after absorbance of cisplatin: 106.5±35.4 nm (50% below 101.1 nm, 90% below 163.3 nm), zeta-potential =−27.9 mV (FIG. 6). Solutions of conjugates remain stably dispersed for periods of up to at least two weeks.

The chemical structure of calcium phosphate nanoparticles prepared in the presence of a dispersing agent was determined by FTIR as follows. Infrared absorption spectra were obtained from calcium phosphate nanoparticles in a KBr pellet using a Bruker Tensor 27 Fourier transform infrared (FTIR) spectrometer with a resolution of 0.1 cm⁻¹. X-ray diffraction analysis was used to determine the crystal structure of the nanoparticles. The samples were scanned with Cu—Kα x-ray radiation from a Philips XRD 2500 at 40 KV and 20 mA, using a step size of 0.02° and a step time of 1.2 s over a 2θ range of 10-70. The particle size of the calcium phosphate nanoparticles and calcium phosphate nanoparticle/cisplatin conjugates were measured on samples dispersed in ultrapure H₂O at about 1 mg/mL concentration by Ultrasonic 1000 L Cell Disruptor. The particle size and Z-potential of nanoparticles was measured on 90 Plus particle sizer coupled with Z-potential analyzer (Brookhaven Instruments, NY, USA).

The FTIR spectra of the nanoparticles prepared in the presence of a dispersing agent and the conjugates have similarities to hydroxyapatite, and not other calcium phosphate phases, except that several peaks associated with the DARVAN® 811 are present in the nanoparticles. However, there is a lack of resolution of the P—O absorption bands, indicating that the sample may contain amorphous calcium phosphate (Legeros Rz et al 2005). The R—COO— stretch in the DARVAN® 811 is changed from 1573 cm⁻¹ to 1559 cm⁻¹ which is possibly due to intermolecular bridge R—COO—Ca complex formation with the CaP. The X-ray diffraction spectra of the nanoparticles prepared in the presence of a dispersing agent contains broad peaks characteristic of hydroxyapatite. The broad peaks of the nanoparticles relative to the hydroxyapatite standard peaks indicates that the crystals are nanometer in size, poorly crystalline or perhaps amorphous. The sample does not show any evidence of contamination from other crystalline calcium phosphate phases.

In vitro cisplatin release studies were conducted by dispersing 40 mg of calcium phosphate nanoparticle/cisplatin conjugates (88 μg/mg loading), by mixing and brief vortexing, in 0.8 mL PBS and rocking at 37° C., 20 cycle/min. Supernatants were collected at 1 hr, 6 hr, 1, 3, 7, 12, and 16 days, after centrifugation at 9,000 rpm (7,000 g) for 10 minutes. The released active agent in the unfiltered supernatant was measured by AAS. Full replacements of release media were made at each time point.

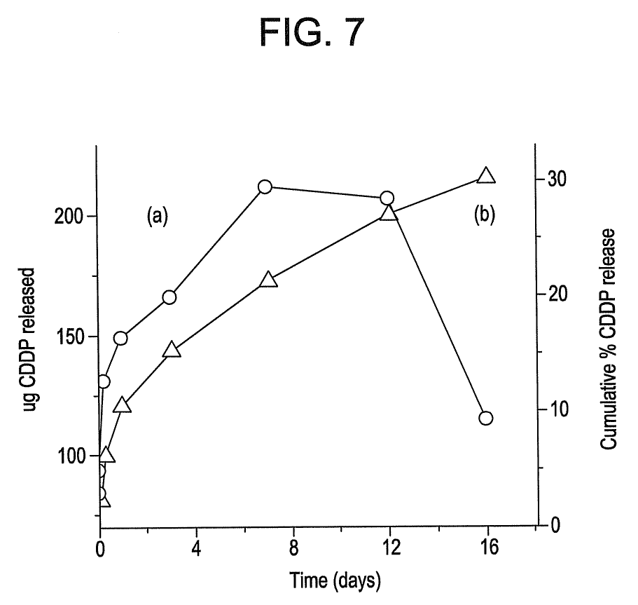

In vitro active agent release from calcium phosphate nanoparticle/cisplatin conjugates: The amount of cisplatin released from the conjugates into PBS, pH=7.4 during gentle rocking at 37° C. at various time points is shown in FIG. 7a. The results are also expressed as a percentage of the total amount bound (FIG. 7b). There is a burst release of active agent in the first day, followed by a slower, but continuous, release of active agent over the time tested. After 16 days in PBS with eight solution changes, 30% of the bound active agent released.

For in vitro cytotoxicity activity studies, the cisplatin-resistant cell line was used: A2780cis human ovarian carcinoma cell line (Sigma, 93112517) and cultured according to supplier's descriptions. Briefly, cells were cultured in RPMI1640 medium, supplemented with 2 mM Glutamine and 10% Fetal Bovine Serum (FBS) in a humid atmosphere at 37° C. and 5% CO₂. Cells were supplemented with 1 μm cisplatin to the culture media every 2-3 passages, post-attachment. The CellTiter96® AQueous One (Promega Corporation, Madison, Wis., USA) colorimetric proliferation assay was used to determine the IC50 value (50% inhibitory concentration) evaluated from 12 two-fold dilutions of cisplatin in 0.9% saline (free active agent), conjugates, calcium phosphate nanoparticle/cisplatin conjugates and free active agent, or cisplatin released from the conjugates. The highest concentrations of test samples were prepared in the active agent master plate prior to dilution as follows: cisplatin was dissolved in 0.9% saline at 1000 μg/mL and diluted in PBS to prepare a free active agent solution of 200 μg cisplatin/mL. Cisplatin released from the conjugates was obtained from the supernatant of 40 mg of conjugates (loaded at 35 μg cisplatin/mg nanoparticles) incubated in 0.8 mL PBS for 3 d on a rocker at 37° C., 20 cycles/min. Three days were necessary to achieve a cisplatin concentration high enough to obtain an IC50 value. Five milligrams of conjugate, synthesized aseptically with a active agent loading of 112 μg cisplatin/mg CaP, was dispersed in 0.8 mL PBS (cisplatin 700 μg/mL if totally released) for the highest conjugate concentration directly added. To confirm that the particle conjugates were diluted evenly across the wells, measurements of the total Pt concentration in all the wells of the active agent master plate were made by AAS after dissolving the conjugate solutions in dilute HCl. Five milligrams of nanoparticles was dispersed in 0.8 mL free active agent solution for the nanoparticles not conjugated to free active agent sample, directly added to cells.

Preliminary investigations of the growth rate of A2780cis were conducted to determine the proper cell seeding number that would remain in the linear range of the assay throughout the study. The cytotoxicity assay was conducted as follows: twenty-four hours after seeding 2000 A2780cis cells in 50 μl of media on 96 well plates, 50 μl PBS, or PBS with active agent, carrier or conjugates was added to the wells. Five replicates were tested for each sample. Following two days of continuous exposure, 20 μl of CellTiter96® AQueous One (Promega) calorimetric proliferation reagent was added to each well, and then the plates were incubated for 4 more hours before being read on a Spectramax Plus384 spectrophotometer (Molecular Biosciences, Sunnyvale, Calif.) at an absorbance value of 490 nm. Absorbance values were converted to IC50 values using the four parameter logistic equation:

Y=(Amax−Amin)/1+(x/IC50)ⁿ+Amin Eq. 2

where Y=observed absorbance

Amax=absorbance of control cells

Amin=absorbance of cells in presence of highest agent concentration

x=active agent concentration (μg/ml)

n=slope of curve

Samples were analyzed for statistically significant differences using the Student's T-test (P<0.05).

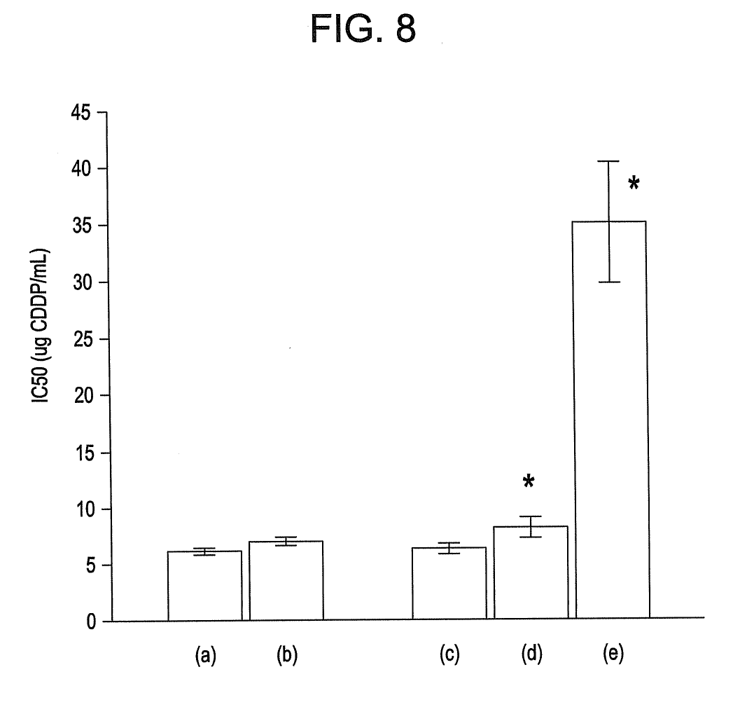

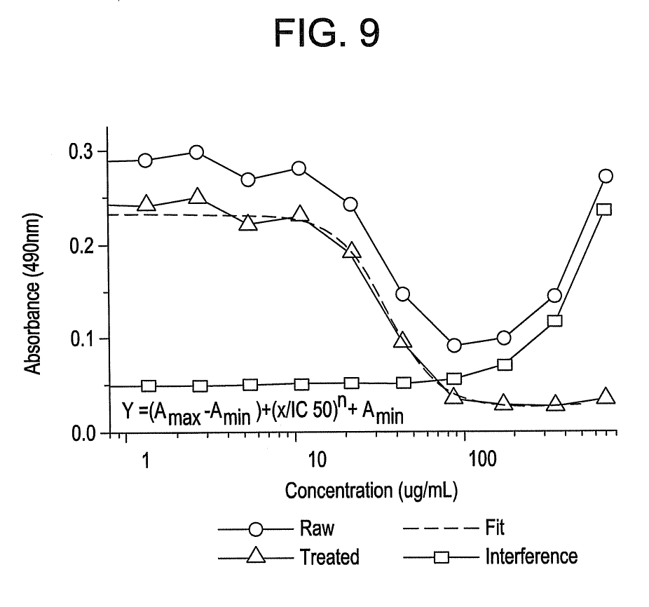

Cytotoxicity of calcium phosphate nanoparticle/cisplatin conjugates: The effect of the cisplatin conjugated to nanoparticles on the proliferation of A2780cis cancer cells was evaluated indirectly and directly by (a) addition of the cisplatin released from the conjugates during incubation in PBS for three days, and (b) direct addition of the conjugates to the cells in culture. The IC50 value obtained for the conjugate-released cisplatin was not significantly different from the free active agent (P>0.05) (FIG. 8), indicating the conjugation procedure and the release process do not adversely affect cisplatin. The IC50 value obtained after direct addition of the conjugates is also shown in FIG. 8. Determination of the IC50 value for directly added conjugates was complicated by the fact that the nanoparticles and the conjugates themselves have an absorbance maximum at 490 nm, the same as the formazan product produced by the viable cells in the assay. Therefore, it was necessary to deduct the interference of the nanoparticles using the readings from wells prepared using the same conditions as above (same seeding cell number, same conjugate or nanoparticle concentration and volume, same culture time) without the addition of CellTiter96® reagent, as shown in FIG. 9. FIG. 9 illustrates the IC50 value determination of calcium phosphate nanoparticle/cisplatin conjugates on A2780Cis cancer cell lines, showing the interference of the nanoparticles and the conjugate particles around 490 nm at higher concentrations. This interference was determined at the same test conditions but without adding Celltiter96 solution. It is subtracted from the raw data to give treated data (treated=raw-interference). The 4-parameter sigmoidal fit of this treated data is used to calculate the IC50 value. The IC50 values obtained this way indicate that the addition of the carrier alone (nanoparticles) to a free active agent solution slightly, but significantly, increases the IC50 value relative to the free active agent alone. This provides indirect evidence that the nanoparticle itself is not cytotoxic at the concentration tested. The IC50 value of the conjugates was found to be significantly higher than the free active agent (35.14±5.33 vs. 6.297±10.43) indicating that a portion of the cisplatin attached to the conjugates is protected from direct interaction with the cells during the two-day test period.

In vitro cytotoxicity testing showed that the cisplatin released from the conjugates retained complete activity during conjugation and release and had comparable cytotoxicity to free active agent. The nanoparticles modified with DARVAN alone was not cytotoxic. Cisplatin release from the conjugates in neutral pH was slow and complete release was limited (30%), therefore the direct addition studies showed reduced cytotoxicity of the conjugated cisplatin relative to free active agent. Particle assisted active agent transport was not a highly active mechanism in this formulation, and not wishing to be bound by theory, the results are possibly due to the negative surface charge or steric stabilization by the DARVAN 811. The surface of the nanoparticle was modified with DARVAN 811 to prevent the adhesion of the nanoparticles to each other through steric stabilization. This modification appears to have also reduced cell membrane adhesion required for CaP particle-assisted active agent transport. Overcoming the repulsive forces may require applying an additional surface modification such as a tumor cell targeting ligand (e.g. folic acid, VEGF, etc.) which will allow cell-specific interactions while maintaining nanoparticle dispersion. However, it is further postulated that in acidic environments such as tumor tissues, the conjugates can slowly dissolve and completely release the adsorbed active agent.

Previous experiments with cisplatin release from different types of calcium phosphate particles that were made without the addition of dispersing agent, slower active agent release from less crystalline CaP was observed. Not wishing to be bound by theory, this effect was correlated to the higher particle surface area of the less crystalline CaP: particles with higher surface areas not only bind more active agent, they release it more slowly and less completely than particles with lower surface areas. The nanoparticles of this study were prepared in the presence of a dispersing agent and appear to be less crystalline than previous experiments due to DARVAN adsorption. This result may explain why the initial burst release and the cumulative active agent release from the conjugates of the present study were lower than that observed previously for CaP not prepared in the presence of a dispersing agent. The reduction of a burst release and enhanced sustained release made possible with the conjugates prepared in the presence of a dispersing agent is desirable for in vivo applications. While there is low cumulative release in neutral PBS, the conjugates are completely soluble in acidic solutions. Active agent loading was verified by totally dissolving the conjugates in 0.1N HCl. This property may make the conjugate delivery system particularly suited for in vivo intratumoral active agent delivery applications in which the acidic pH of tumor tissue will lead eventually to complete active agent release and dissolution of the inorganic particles.

The reduced cytotoxicity of the conjugates prepared in the presence of a dispersing agent relative to free active agent seen in the direct addition studies also confirms that a large portion of cisplatin attached to the CaP is not released over two days in neutral pH cell culture medium. From the in vitro release studies using conjugates made with CaP prepared in the presence of a dispersing agent (FIG. 7), approximately 12% of the total cisplatin bound would be expected to be released by the end of the two day incubation with cells. The 12% is probably an overestimate since the release study was conducted with solution agitation and multiple total solution replacements, while the cell culture media was not disturbed or replaced. Assuming 10% release, of the 350 μg/ml available for release from the conjugates, only 35 μg/ml of free active agent would have been available at the highest concentration to the cells compared to the 100 μg/ml in the free active agent wells. If the concentrations of the conjugates were adjusted for the IC50 determination, the IC50 would be 3.5 μg/mL, which is less than free active agent (6.3 μg/mL). Therefore is appears that more of the cisplatin than expected from cell-free in vitro release studies is being released when in contact with cells. This would be possible if some endocytosis of the conjugates occurred.

### Example 3

### Comparison of Calcium Phosphate Nanoparticle Prepared in the Presence of a Dispersing Agent and Calcium Phosphate Particles, Micrometer-Sized

Calcium phosphate nanoparticles were compared to calcium phosphate micrometer-sized particles for their in vitro active agent release properties. Calcium phosphate nanoparticles were prepared by the addition of Darvan during the calcium phosphate precipitation similar to Example 2 above. After a 1 hr reaction time, dispersed 119 nm particulates were collected. The micro- and nanoparticles were characterized TEM, FTIR, XRD, particle size analysis and zeta potential measurements. Complexes of the calcium phosphate particles and cisplatin were prepared through electrostatic binding of an aquated species of cisplatin to the calcium phosphate particles in a chloride-free phosphate buffer. The active agent loading was determined by platinum atomic absorption spectroscopy. Active agent release studies were completed at 4 hours, 1 day, 3 days, 7 days, 10 days and 15 days. The nanoparticles released only slightly more active agent over the 15 day release assay (53% of the loaded active agent vs. 40%) as compared to the micrometer-sized complexes; however, there was desirable reduction in the active agent burst release and an extension of the sustained release. The toxicity of the released active agent from both types of conjugates was compared to that of the free cisplatin in vitro with the CellTiter cell proliferation assay using a mouse carcinoma cell line. IC50 values of both conjugates were found to be statistically equivalent to pure cisplatin indicating no adverse reaction between the cisplatin and the nanoparticles. Furthermore, nano-sizing the complexes increased the injectibility from an 18 gauge to a 26 gauge needle.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All ranges disclosed herein are inclusive and combinable.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms, and any polymorphs of the compound are contemplated herein. Compounds may contain an asymmetric element such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

The essential characteristics of the present invention are described completely in the foregoing disclosure. One skilled in the art can understand the invention and make various modifications without departing from the basic spirit of the invention, and without deviating from the scope and equivalents of the claims, which follow. Moreover, any combination of the above-described elements in all possible variations thereof is encompassed by the invention unless otherwise indicated herein or otherwise clearly contradicted by context.

What is claimed is:

1. An active agent delivery system, comprising:
a calcium phosphate nanoparticle active agent conjugate comprising an active agent adsorbed onto calcium phosphate nanoparticles, wherein the calcium phosphate nanoparticles are prepared with a dispersing agent; and wherein a calcium phosphate nanoparticle active agent conjugate further comprises a targeting ligand.

2. The active agent delivery system of claim 1, wherein the calcium phosphate nanoparticle is amorphous calcium phosphate.

3. The active agent delivery system of claim 1, wherein the calcium phosphate nanoparticle is hydroxyapatite.

4. The active agent delivery system of claim 1, wherein the calcium phosphate nanoparticles are prepared by precipitation in the presence of the dispersing agent.

5. The active agent delivery system of claim 1, wherein the calcium phosphate nanoparticles have a mean particle diameter of about 10 to about 100 nanometers.

6. The active agent delivery system of claim 1, wherein the calcium phosphate nanoparticles have a mean particle diameter of about 100 to about 300 nanometers.

7. The active agent delivery system of claim 1, wherein the dispersing agent is a polyelectrolyte, a surfactant, a polysaccharide, a carbohydrate, an amino acid, a polyamino acid, a poloxamer, gelatin, a polyethylene glycol, an acrylic-based polymeric salt, or a combination comprising at least one of the foregoing dispersing agents.

8. The active agent delivery system of claim 1, wherein the dispersing agent is poly(allylamine hydrochloride), heparin, L-aspartic acid, lysine, glycine, poly-L-lysine, or a combination comprising at least one of the foregoing dispersing agents.

9. The active agent delivery system of claim 1, wherein the dispersing agent is a sodium polyacrylate having a M_{W} of about 2000 to about 5000, a sodium polymethacrylate having a M_{W} of about 2000 to about 5000, or a combination comprising at least one of the foregoing dispersing agents.

10. The active agent delivery system of claim 1, wherein the targeting ligand is an antibody, a vitamin, a protein, an amino acid, polyamino acid, or a combination comprising at least one of the foregoing targeting ligands.

11. The active agent delivery system of any one of claim 1, wherein the targeting ligand is folic acid or vascular endothelial growth factor.

12. The active agent delivery system of claim 1, wherein the active agent is an alpha-2 adrenergic agent, an analgesic, an angiotensin-converting enzyme (ACE) inhibitor, an antianxiety agent, an antiarrhythmic, an antibacterial, an antibiotic, an anticancer agent, an antidepressant, an antidiabetic, an antiepileptic, an antifungal antihelminthic, an antihyperlipidemic, an antihypertensive agent, an antiinfective, an antimalarial, an antimicrobial, an antimigraine agent, an antimuscarinic agent, an antineoplastic agent, an antiprotozoal agent, an antipsychotic agent, an antispasmodic, an antiviral agent, an attention-deficit hyperactivity disorder (ADHD) agent, a β-blocker, a calcium channel blocker, a chemotherapeutic agent, a cholinesterase inhibitor, a Cox-2 inhibitor, a hypnotic, a hypotensive agent, an immunosuppressant, a lipotropic, a neuroleptic, an opioid analgesic, a peripheral vasodilator/vasoconstrictor, a sedative, or a serotonin receptor agonist.

13. The active agent delivery system of claim 1, wherein the active agent is aminoglutethimide, busulfan, carmustine, chlorambucil, cisplatin, cyclophosphamide, cytarabine, dacarbazine, daunorubicin, diethylstilbestrol, doxorubicin, etoposide, fluorouracil, fluoxymesterone, flutamide, gemcitabine, goseraline acetate, hydroxyprogesterone, hydroxyurea, leuprolide, lomustine, mechlorethamine, medroxyprogesterone acetate, megestrol acetate, melphalan, mercaptopurine, methotrexate, paclitaxel, prednisone, procarbazine, tamoxifan, testosterone propionate, thioguanine, vinblastine, vincristine, vindesine, vinorelbine, a pharmaceutically acceptable salt thereof, or a combination comprising at least one of the foregoing anticancer agents.

14. The active agent delivery system of claim 1, wherein the active agent is cisplatin or aquated cisplatin.

15. A method to treat or prevent a disease condition in a patient, comprising:
administering the system of claim 1 to a patient in need thereof.

16. A method to treat cancer, comprising:
administering the system of claim 1 to a patient in need thereof.

17. The method of claim 16, further comprising radiotherapy, surgery, systemic chemotherapy, or a combination comprising at least one of the foregoing.

18. A method to treat or prevent cancer metastasis, comprising: administering the system of any one of claim 1 to a patient in need thereof.

19. The method of claim 18, wherein the calcium phosphate nanoparticle active agent conjugate accumulates in the lymph nodes of the patient.
